(19) United States
(12) Patent Application Publication
(10) Pub. No.: US 2026/0234635 A1
(43) Pub. Date: Aug. 13, 2026

(54) COMPOSITIONS AND METHODS FOR THE TREATMENT OF SEPSIS

(71) Applicant: John Mansell, Gillette, WY (US)

(72) Inventor: John Mansell, Gillette, WY (US)

(21) Appl. No.: 19/156,611

(22) PCT Filed: Feb. 15, 2024
(86) PCT No.: PCT/US2024/015901

§ 371 Date: Aug. 14, 2025

Related U.S. Application Data
(60) Provisional application No. 63/445,789, filed on Feb. 15, 2023.

(51) Int. Cl. C12N 15/113; A61K 45/06; C07K 16/18; C07K 16/26; C07K 16/40

(52) U.S. Cl. CPC C12N 15/1137; A61K 45/06; C07K 16/18; C07K 16/26; C07K 16/40; C12N 15/1136; C12Y 114/13039; C12Y 114/99001; C12Y 304/24; C12Y 401/01022

(57) ABSTRACT

Compositions disclosed herein comprise materials which (i) reduce the amount of one or more nitric oxide synthases present; (ii) reduce the level of expression of one or more nitric oxide synthases; and/or (iii) reduce the catalytic activity of one or more nitric oxide synthases is referred to herein as a nitric oxide nNOS disruptor (NOD).

### CROSS-REFERENCE TO RELATED APPLICATIONS

This application claims priority to U.S. Provisional Patent Application Ser. No. 63/445,789 filed Feb. 15, 2023 and entitled “Compositions and Methods for the Treatment of Sepsis.”

### FIELD

The present disclosure relates generally to therapeutic agents for the treatment of disease states. More particularly, the present disclosure relates to methods and compositions for the treatment of sepsis.

### BACKGROUND

Sepsis is a body-wide inflammatory and hyperemic multifactorial response to a noxious trigger, trauma, toxin, or infection. The global burden of sepsis is difficult to ascertain, although a recent scientific publication estimated that in 2017 there were 48.9 million cases and 11 million sepsis-related deaths worldwide annually, which accounted for almost 20% of all global deaths. In 2017, almost half of all global sepsis cases occurred among children, with an estimated 20 million cases and 2.9 million global deaths in children under five years of age. Significant regional disparities in sepsis incidence and mortality exist; approximately 85.0% of sepsis cases and sepsis-related deaths worldwide occurred in low-and middle-income countries.

During sepsis, over-expression of cytokines or the systemic inflammatory response causes (i) hypotension, (ii) impaired oxygen delivery to tissues and (iii) parenchymal edema with distortion of tissue architecture in organs. These and other factors that occur during sepsis result in diminished organ function typically leading to multiple organ failure.

Because sepsis results in an over-expression of the pathogenic compounds produced by several different systems, inhibiting any one system has not proven to be clinically effective. The redundancy of defensive mechanisms that has helped to preserve our species from regional threats to the body, say, an infection in your hand, fails when this system is switched on body-wide.

Historically clinical treatment of sepsis has been restricted to finding and treating the trigger while mitigating the symptoms of sepsis. For example, the debridement of burns, clearance of a toxin, or finding and administering an effective antibiotic or antiviral are among the measures that could terminate the septic trigger, but the septic race is for these actions to take effect before the patient expires or permanent organ damage is done.

While infusions of drugs to treat sepsis use membrane receptors which trigger an intracellular response, no clinical treatment thus far addresses nuclear and cytoplasmic responses directly. No treatment has directly addressed the over-expression of proteins and enzymes that are the causes of the symptoms of sepsis. An ongoing need exists for the development of methods and compositions able to effectively address the therapeutic needs of individuals suffering from sepsis.

### SUMMARY

The present disclosure includes one or more of the features recited in the appended claims and/or the following features, which, alone or in any combination, may comprise patentable subject matter.

According to a first aspect of the disclosed embodiments of methods and compositions for the treatment of sepsis, a composition comprising at least two, and as many as all, materials is selected from a material group. The material group consists of (a) a neuronal nitric oxide synthase silencing nucleotide; (b) a neuronal nitric oxide synthase binding antibody; (c) an endothelial nitric oxide synthase silencing nucleotide; (d) an endothelial nitric oxide synthase binding antibody; (e) an inducible nitric oxide synthase silencing nucleotide; (f) an inducible nitric oxide synthase binding antibody; (g) a calcitonin gene-related peptide silencing nucleotide; (h) a calcitonin gene-related peptide binding antibody; (i) a disintegrin and metalloprotease peptide silencing nucleotide; (j) a disintegrin and metalloprotease binding antibody; (k) a bradykinin peptide silencing nucleotide; (1) a bradykinin binding antibody; (m) a substance P peptide silencing nucleotide; (n) a substance P binding antibody; (o) a histidine decarboxylase silencing nucleotide; (p) a histidine decarboxylase binding antibody; (q) an antihistamine; (r) a bradykinin antagonist; (s) a cyclooxygenase silencing nucleotide; (t) a cyclooxygenase binding antibody; (u) a small molecule prostacyclin inhibitor; (v) a prostacyclin synthase silencing nucleotide; and (w) a prostacyclin synthase binding antibody.

In some embodiments of the first aspect, at least one of the (b), (d), (f), (h), (1), (n), (p), (t) or (w) materials is from the IgG family of immunoglobulins.

Optionally, in the first aspect, at least one of the (b), (d), (f), (h), (l), (n), (p), (t) or (w) materials is a functional fragment.

According to a second aspect of the disclosed embodiments of methods and compositions for the treatment of sepsis, a medicament comprising a composition, wherein the composition comprises at least two, and as many as all, materials are selected from a material group. The material group consists of (a) a neuronal nitric oxide synthase silencing nucleotide; (b) a neuronal nitric oxide synthase binding antibody; (c) an endothelial nitric oxide synthase silencing nucleotide; (d) an endothelial nitric oxide synthase binding antibody; (e) an inducible nitric oxide synthase silencing nucleotide; (f) an inducible nitric oxide synthase binding antibody; (g) a calcitonin gene-related peptide silencing nucleotide; (h) a calcitonin gene-related peptide binding antibody; (i) a disintegrin and metalloprotease peptide silencing nucleotide; (j) a disintegrin and metalloprotease binding antibody; (k) a bradykinin peptide silencing nucleotide; (l) a bradykinin binding antibody; (m) a substance P peptide silencing nucleotide; (n) a substance P binding antibody; (o) a histidine decarboxylase silencing nucleotide; (p) a histidine decarboxylase binding antibody; (q) an antihistamine; (r) a bradykinin antagonist; (s) a cyclooxygenase silencing nucleotide; (t) a cyclooxygenase binding antibody; (u) a small molecule prostacyclin inhibitor; (v) a prostacyclin synthase silencing nucleotide; and (w) a prostacyclin synthase binding antibody.

According to a third aspect of the disclosed embodiments of methods and compositions for the treatment of sepsis, a method comprises the administration of a medicament comprising a composition, wherein the composition comprises at least two, and as many as all, materials are selected from a material group. The material group consists of (a) a neuronal nitric oxide synthase silencing nucleotide; (b) a neuronal nitric oxide synthase binding antibody; (c) an endothelial nitric oxide synthase silencing nucleotide; (d) an endothelial nitric oxide synthase binding antibody; (e) an inducible nitric oxide synthase silencing nucleotide; (f) an inducible nitric oxide synthase binding antibody; (g) a calcitonin gene-related peptide silencing nucleotide; (h) a calcitonin gene-related peptide binding antibody; (i) a disintegrin and metalloprotease peptide silencing nucleotide; (j) a disintegrin and metalloprotease binding antibody; (k) a bradykinin peptide silencing nucleotide; (l) a bradykinin binding antibody; (m) a substance P peptide silencing nucleotide; (n) a substance P binding antibody; (o) a histidine decarboxylase silencing nucleotide; (p) a histidine decarboxylase binding antibody; (q) an antihistamine; (r) a bradykinin antagonist; (s) a cyclooxygenase silencing nucleotide; (t) a cyclooxygenase binding antibody; (u) a small molecule prostacyclin inhibitor; (v) a prostacyclin synthase silencing nucleotide; and (w) a prostacyclin synthase binding antibody.

According to a fourth aspect of the disclosed embodiments of methods and compositions for the treatment of sepsis, a medicament comprising a composition is used to reduce expression of sepsis-related pathogenic compounds, wherein the composition comprises at least two, and as many as all, materials are selected from a material group. The material group consists of (a) a neuronal nitric oxide synthase silencing nucleotide; (b) a neuronal nitric oxide synthase binding antibody; (c) an endothelial nitric oxide synthase silencing nucleotide; (d) an endothelial nitric oxide synthase binding antibody; (e) an inducible nitric oxide synthase silencing nucleotide; (f) an inducible nitric oxide synthase binding antibody; (g) a calcitonin gene-related peptide silencing nucleotide; (h) a calcitonin gene-related peptide binding antibody; (i) a disintegrin and metalloprotease peptide silencing nucleotide; (j) a disintegrin and metalloprotease binding antibody; (k) a bradykinin peptide silencing nucleotide; (l) a bradykinin binding antibody; (m) a substance P peptide silencing nucleotide; (n) a substance P binding antibody; (o) a histidine decarboxylase silencing nucleotide; (p) a histidine decarboxylase binding antibody; (q) an antihistamine; (r) a bradykinin antagonist; (s) a cyclooxygenase silencing nucleotide; (t) a cyclooxygenase binding antibody; (u) a small molecule prostacyclin inhibitor; (v) a prostacyclin synthase silencing nucleotide; and (w) a prostacyclin synthase binding antibody.

### BRIEF DESCRIPTION OF THE DRAWING

FIG. 1 illustrates three isoforms of nitric oxide synthase-neuronal NOS (nNOS), endothelial NOS (eNOS), and inducible NOS (iNOS).

### DETAILED DESCRIPTION

Disclosed herein are methods and compositions for the treatment of one or more symptoms associated with the condition of sepsis. In an aspect, the methods disclosed herein include administration of compositions of the type disclosed herein to subjects presenting with sepsis. Alternatively, compositions of the type disclosed herein are administered to subjects presenting with septic shock. Herein sepsis is defined as a life-threatening organ dysfunction caused by a dysregulated host response to infection. Herein septic shock refers to sepsis with circulatory and cellular/metabolic abnormalities profound enough to substantially increase mortality. In one or more aspects, a subject administered one or more compositions of the present disclosure has a suspected or documented case of sepsis and an acute increase of equal to or greater than about 2 sequential organ failure assessment (SOFA) points which is a proxy for organ dysfunction.

In one or more aspects, a subject is considered clinically to be in septic shock when sepsis and vasopressor therapy is needed to elevate the mean arterial pressure to equal to or greater than about 65 mm Hg and the subject's lactate concentration is greater than about 2 mmol/L (18 mg/dL) after adequate fluid resuscitation. It is contemplated the various actions that result from administration of one or more of the presently disclosed compositions result in the described events (e.g., reduction in the amount of one or more nitric oxide synthases) beneficially affecting the septic state of the subject such as be alleviating symptoms associated with the disorder.

Other materials for use in the methods of the present disclosure will be described in more detail later herein. Methodologies of the present disclosure utilize at least two materials that are able to effectively reduce the expression of sepsis mediators in order to mitigate the physiologically adverse events associated with sepsis. Compositions of the present disclosure may be further characterized as comprising materials whose presence as intact functioning compounds in a physiological environment (e.g., half-life in the bloodstream, time for metabolite removal) is less than about 48 hours, alternatively less than about 36 hours, or alternatively less than about 24 hours.

In an aspect, administration of the compositions disclosed herein to a subject suffering from sepsis results in an increase in the therapeutic time window of the subject by greater than about 10%, alternatively greater than about 25% or alternatively greater than about 50%. Herein the “therapeutic time window” refers to the time range between injury and treatment during which the treatment is still effective. Compositions of the present disclosure comprising materials that transiently extend the therapeutic time window are hereinafter designated window-extending therapies or WETs.

In an aspect, the WET comprises a material that (i) reduces the amount of one or more nitric oxide synthases present in the body; (ii) reduces the level of expression of one or more nitric oxide synthases; and/or (iii) reduces the catalytic activity of one or more nitric oxide synthases is referred to herein as a nitric oxide disruptor (NOD).

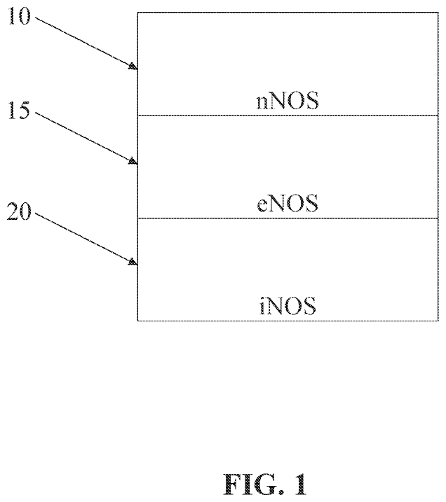

Nitric oxide (NO), the smallest signaling molecule known, is produced by, as shown in FIG. 1, three isoforms of NO synthase (NOS; EC 1.14.13.39), neuronal NOS (shown in block 10), endothelial NOS (shown in block 15) and inducible NOS (shown in block 20). All three isoforms utilize L-arginine and molecular oxygen as substrates and require the cofactors reduced nicotinamide-adenine-dinucleotide phosphate (NADPH), flavin adenine dinucleotide (FAD), flavin mononucleotide (FMN), and (6R-)5,6,7,8-tetrahydrobioptcrin (BH₄).

Neuronal NOS (nNOS, NOS I) is constitutively expressed in central and peripheral neurons and some other cell types. The enzyme has 1434 amino acids and exists as dimer in its active form. Each monomer of nNOS consists of two domains: N-terminal (catalytic or oxygenase) and C-terminal (reductase) where the N-terminal domain binds to the thiolate-linked heme group, tetrahydrobiopterin (BH4), a redox co-factor; L-arginine the substrate, and the zinc ion. The C-terminal domain has binding sites for flavin mononucleotide (FMN), flavin adenine dinucleotide (FAD), and nicotinamide adenine dinucleotide phosphate (NADPH).

Endothelial NOS (eNOS, NOS III) is mostly expressed in endothelial cells, eNOS functions to keep blood vessels dilated, control blood pressure, and has numerous other vasoprotective and anti-atherosclerotic effects. Many cardiovascular risk factors lead to oxidative stress, eNOS uncoupling, and endothelial dysfunction in the vasculature.

Inducible NOS (iNOS) contributes to the pathophysiology of septic shock. iNOS is not usually expressed in cells, but its expression can be induced by bacterial lipopolysaccharide, cytokines, and other agents. Although primarily identified in macrophages; expression of iNOS can be stimulated in virtually any cell or tissue, provided that the appropriate inducing agents have been identified. Once expressed, iNOS is constantly active and not regulated by intracellular Ca²⁺ concentrations. Excessive NO production by iNOS plays a crucial role in septic shock. This condition is characterized by massive arteriolar vasodilatation, hypotension, and microvascular damage with the fall in blood pressure predominantly due to excess NO production by iNOS induced in the vascular wall.

In one or more aspects of the present disclosure, the WET comprises an antibody that binds to and neutralizes one or more isotopes of NOS. Herein neutralize refers to the ability to bind to and diminish or remove the function, catalytic activity or both of the bound molecule. For example, a WET may comprise a neutralizing antibody that binds iNOS, nNOS, eNOS, or any combination thereof. In an aspect, the neutralizing antibody binds iNOS with a binding affinity that is equal to or greater than about 2×, alternatively equal to or greater than about 5× or alternatively equal to or greater than about 10×0 the binding affinity of an antibody for nNOS or eNOS. In an alternative aspect, the neutralizing antibody binds iNOS with a binding affinity that is equal to or greater than about 2×, alternatively equal to or greater than about 5× or alternatively equal to or greater than about 10× the binding affinity of an antibody for eNOS.

In an aspect, the WET further comprises one or more siRNAs to arrest or diminish expression of iNOS thus preventing septic hypotension Additionally, because there is almost always a background or basal expression of proteins in the body, a septic trigger may cause enzyme activation of the iNOS proteins already in existence before an episode of sepsis formally starts. Since these “pre-sepsis” enzymes contribute to hypotension, administration of domain-specific iNOS neutralizing antibodies could arrest their function. Consequently, a method of the present disclosure comprises treating a subject diagnosed or suspected of having sepsis with a WET comprising a fast-acting iNOS antibody (AbiNOS). Without wishing to be limited by theory, the use of a (AbiNOS) would reduce or eliminate a clinical lag time characterized by hypotension while waiting on existing iNOS enzymes to stop functioning.

Inhibition of expression of one or more NOS with an siRNA as well as arresting function with a domain-specific NOS antibody would attenuate release of NO during sepsis. In an aspect, the WET comprises both an antibody that binds one or more NOS in order to reduce the endogenous level of NO and an siRNA that reduces expression of one or more genes associated with the production of NO.

In an aspect, the WET comprises a material that (i) reduces the amount of tumor necrosis factor (TNF); (ii) reduces the level of expression of enzymes that release tumor necrosis factor; and/or (iii) reduces the catalytic activity of enzymes that release tumor necrosis factor. A method of the present disclosure may comprise administration of one or more antibodies that affect the production of tumor necrosis factor. For example, the materials may comprise an antibody that upon binding removes the function, catalytic activity or both of the TNF-generating disintegrin and metalloprotease, ADAM17.

ADAM17 is a metalloproteinase that facilitates shedding of TNF, a cytokine that contributes to the systemic inflammatory response characteristic of sepsis. Inhibition of expression of ADAM17 with an siRNA as well as arresting function with a domain-specific ADAM17 antibody would attenuate release of TNF and other cytokines during sepsis. In an aspect, the WET comprises both an antibody that binds ADAM17 in order to reduce the endogenous level of TNF and an siRNA that reduces expression of one or more genes associated with the production of TNF (e.g., siRNA to ADAM17).

In an aspect, the WET further comprises a material that (i) reduces the amount of calcitonin gene-related peptide (CGRP); (ii) reduces the level of expression of CGRP; and/or (iii) reduces the catalytic activity of calcitonin gene-related peptide (CGRP). A method of the present disclosure may comprise administration of a WET comprising one or more antibodies that neutralize CGRP. For example, the materials may comprise an antibody that removes the function, catalytic activity or both of the bound molecule of CGRP. CGRP is a polypeptide that among its other effects, causes vasodilation. Without wishing to be limited by theory, inhibiting expression of CGRP during sepsis would remove another systemic response causing hypotension with simultaneous use of CGRP antibodies would interfere with existing messenger molecules (i.e., basal level of CGRP present). In an aspect, the WET comprises both an antibody that binds CGRP in order to reduce the endogenous level of CGRP and an siRNA that reduces expression of one or more genes associated with the production of CGRP.

In an aspect, the WET further comprises a material that (i) reduces the amount of bradykinin (BK); (ii) reduces the level of expression of BK; and/or (iii) reduces the catalytic activity of BK. BK is a nine-amino acid polypeptide that causes histamine release, vasodilation, increased vascular permeability, is pro-inflammatory and triggers TNF release. The materials for use in the WET may comprise an antibody that removes the function, catalytic activity or both of the bound molecule of BK. Without wishing to be limited by theory, inhibiting expression of BK during sepsis would remove another systemic response causing hypotension with simultaneous use of BK antibodies that would interfere with existing messenger molecules. In an aspect, the WET comprises both an antibody that binds BK in order to reduce the endogenous level of BK and an siRNA that reduces expression of one or more genes associated with the production of BK.

In an alternative aspect, the WET comprises a BK antagonist. Such BK antagonists are expected to function as anti-inflammatory and analgesic agents. BK antagonists that may be suitably included in the WET include any BK antagonist compatible with the other components of the WET. Nonlimiting examples of BK antagonists suitable for use in the present disclosure include icatibant and fastibant.

In an aspect, the WET further comprises a material that (i) reduces the amount of Substance P; (ii) reduces the level of expression of Substance P; and/or (iii) reduces the function of Substance P. Substance P is an cleven amino acid neuropeptide that enhances the effects of BK, is vasodilatory and initiates expression of cytokines. The WET may comprise, an antibody that removes the function, catalytic activity or both of the bound molecule of Substance P. Without wishing to be limited by theory, inhibiting expression of Substance P during sepsis would remove another systemic response causing hypotension with simultaneous use of Substance P antibodies that would interfere with existing messenger molecules. In an aspect, the WET comprises both an antibody that binds Substance P in order to reduce the endogenous level of Substance P and an siRNA that reduces expression of one or more genes associated with the production of Substance P.

In an aspect, the WET further comprises a material that (i) reduces the amount of histamine; (ii) reduces the level of expression of at least one histamine-producing molecule; and/or (iii) reduces the function of at least one histamine-producing molecule. In one or more aspects, histamine production is reduced by administration of one or more conventional antihistamines. Examples of conventional antihistamines include, without limitation, carbinoxamine maleate, brompheniramine, chlorpheniramine, clemastine, diphenhydramine, hydroxyzine, triprolidine, cetirizine, and fexofenadine.

Additionally or alternatively, inhibition of histamine production or the reduction of histamine levels is achieved by decreasing the levels and/or activity of the enzyme, L-histidine decarboxylase (EC 4.1. 1.2, HDC). For example, a WET of the present disclosure may comprise an antibody that removes the function, catalytic activity or both of a bound HDC.

Without wishing to be limited by theory, inhibiting expression of HDC during sepsis would remove another systemic response observed during sepsis with simultaneous use of HDC antibodies that would interfere with existing messenger molecules. In an aspect, the WET comprises both an antibody that binds HDC in order to reduce the endogenous level of HDC and an siRNA that reduces expression of one or more genes associated with the production of HDC.

In an aspect, the WET further comprises a material that (i) reduces the amount of prostacyclin (PgI₂); (ii) reduces the level of expression of biomolecules involved in the production of PgI₂; and/or (iii) reduces the catalytic activity of biomolecules involved in the production of PgI₂. Prostacyclin is a member of the prostaglandin family of bioactive lipids. Upon the release of prostacyclin from epithelial cells of the vascular wall, the lipid functions as a potent vasodilator and inhibitor of platelet aggregation. Prostacyclin is synthesized from arachidonic acid via the actions of prostacyclin synthase (PS) and cyclooxygenase enzymes (COX). In one or more aspects, inhibition of prostacyclin levels is achieved by decreasing the levels and/or activity of the enzymes involved in the production of prostacyclin such as PS or COX. For example, a WET of the present disclosure may comprise one or more antibodies that inhibits the function, catalytic activity or both of a bound PS, a bound COX or both. Additionally or alternatively, of the present disclosure may comprise one or more siRNA that reduces the expression of PS, COX or both

Methods and compositions for the RNA-mediated silencing of sepsis-related proteins are described in the patent entitle Methods and Compositions for Sepsis-Related Disorders, U.S. Patent Publication Number 2020/20200276223 to Mansell; the entirety of which is incorporated by reference herein.

In one or more aspects of the present disclosure, the antibody molecule utilized in a WET of the present disclosure binds to a mammalian biomolecule of the type disclosed herein (e.g., human, iNOS). For example, the antibody molecule binds specifically to an epitope, e.g., linear or conformational epitope of a biomolecule of the type disclosed herein.

As used herein, the term “antibody molecule” refers to a protein, e.g., an immunoglobulin chain or fragment thereof, comprising at least one immunoglobulin variable domain sequence. The term “antibody molecule” includes, for example, a monoclonal antibody (including a full length antibody which has an immunoglobulin Fe region). In an aspect, an antibody molecule comprises a full length antibody, or a full length immunoglobulin chain. In an aspect, an antibody molecule comprises an antigen binding or functional fragment of a full length antibody, or a full length immunoglobulin chain. Consequently, the term antibody as used herein refers to intact molecules as well as functional fragments thereof.

Constant regions of the antibodies can be altered, e.g., mutated, to modify the properties of the antibody (e.g., to increase or decrease one or more of: Fe receptor binding, antibody glycosylation, the number of cysteine residues, effector cell function, or complement function).

In an aspect, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. Herein an epitope refers to the part of an antigen molecule to which an antibody attaches itself. Herein an antigen refers to any molecule that can bind specifically to an antibody.

In an aspect, an antibody molecule is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an aspect, the first and second epitopes are on the same antigen, e.g., the same protein (or subunit of a multimeric protein). In another aspect the first and second epitopes overlap. In yet another aspect, the first and second epitopes do not overlap. In an aspect, the first and second epitopes are on different antigens, in other words different proteins (e.g., iNOS and CGRP). In an aspect, a multispecific antibody molecule comprises a third, fourth or fifth immunoglobulin variable domain. In an aspect, a multispecific antibody molecule is a bispecific antibody molecule, a trispecific antibody molecule, or tetraspecific antibody molecule, In an aspect, an antibody molecule comprises a diabody, and a single-chain molecule, as well as an antigen-binding fragment of an antibody (e.g., Fab, F(ab′)2, and Fv). For example, an antibody molecule can include a heavy (H) chain variable domain sequence (abbreviated herein as VH), and a light (L) chain variable domain sequence (abbreviated herein as VL). In an aspect an antibody molecule comprises or consists of a heavy chain and a light chain (referred to herein as a half antibody). In another example, an antibody molecule includes two heavy (H) chain variable domain sequences and two light (L) chain variable domain sequences, thereby forming two antigen binding sites, such as Fab, Fab′, F(ab′)2, Fe, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de nova using recombinant DNA technologies. Such antibodies for use in the methods and compositions disclosed herein may comprise functional antibody fragments that retain the ability to selectively bind with their respective antigen or receptor.

The preparation of antibody molecules can be monoclonal or polyclonal. An antibody molecule for use in the present disclosure may also be a human, humanized, CDR-grafted, or in vitro generated antibody. In an aspect, the antibody or antibody fragment has a heavy chain constant region from the IgG class (e.g., IgG 1, IgG2, IgG3, or IgG4). The antibody can also have a light chain chosen from, e.g., kappa or lambda. The term “immunoglobulin” (lg) is used interchangeably with the term “antibody” herein.

Examples of antigen-binding fragments of an antibody molecule include: (i) a Fab fragment, a monovalent fragment consisting of the VL, VH, CL and CHI domains; (ii) a F(ab′)2 fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); and (viii) a single domain antibody. These antibody fragments are obtained using conventional techniques known to those with skill in the art, and the fragments are screened for utility in the same manner as are intact antibodies.

The terms “treat,” “treating,” or “treatment,” as used herein, include alleviating, abating, or ameliorating a disease or condition, or symptoms thereof; managing a disease or condition, or symptoms thereof; preventing additional symptoms; ameliorating or preventing the underlying metabolic causes of symptoms; inhibiting the disease or condition, e.g., arresting the development of the disease or condition; relieving the disease or condition; causing regression of the disease or condition; relieving a symptom caused by the disease or condition; stopping the symptoms of the disease or condition; and combination of these. Treatment as used herein also encompasses any pharmaceutical or medicinal use of the compositions herein.

The term “subject” as used herein, refers to an animal which is the object of treatment, observation, or experiment. By way of example only, a subject may be, but is not limited to, a mammal including, but not limited to, a human. In an aspect, the subject is administered the compositions disclosed herein in a therapeutically effective amount sufficient for treating, preventing, and/or amcliorating one or more symptoms of sepsis. As used herein, amelioration of a symptom of sepsis by administration of a particular composition of the type disclosed herein refers to any lessening of that symptom, whether lasting or transient, which can be attributed to or associated with administration of compositions of the type disclosed herein. It is contemplated that the therapeutically effective amount may be optimized by one or more healthcare professionals in consideration of the particular factors affecting a subject.

As used herein, the term “RNA interference” or “RNAi” refers to the silencing or decreasing of gene expression by iRNA agents (e.g., siRNAs, miRNAs, shRNAs), via the process of sequence-specific, post-transcriptional gene silencing in animals and plants, initiated by an iRNA agent that has a seed region sequence in the iRNA guide strand that is complementary to a sequence of the silenced gene. As used herein, the term an “iRNA agent” (abbreviation for “interfering RNA agent”), refers to an RNA agent, or chemically modified RNA, which can down-regulate the expression of a target gene. The phrase “chemical modification” as used herein refers to that meaning as is generally accepted in the art. When used with reference to the nucleic acid molecules of the present disclosure, “chemical modification” refers to any modification of the chemical structure of the nucleotides such that the resultant chemical structure differs from that of the nucleotides of native siRNA or RNA in general. For example, the term “chemical modification” encompasses the addition, substitution, or modification of native siRNA or RNA at the sugar, base, or internucleotide linkage, as described herein or as is otherwise known in the art. In certain aspects, the term “chemical modification” can refer to certain forms of RNA that are naturally occurring in certain biological systems, for example 2′-0-methyl modifications or inosine modifications. While not wishing to be bound by theory, an iRNA agent may act by one or more of a number of mechanisms, including post-transcriptional cleavage of a target mRNA, or pre-transcriptional or pre-translational mechanisms. An iRNA agent can be single-stranded (ss) or can include more than one strand, e.g., it can be a double-stranded (ds) iRNA agent. As used herein, the term “siRNA” refers to a small interfering RNA. siRNAs include short interfering RNA of about 15-60, 15-50, or 15-40 (duplex) nucleotides in length, more typically about 15-30, 15-25 or 19-25 (duplex) nucleotides in length (e.g., each complementary sequence of the double stranded siRNA is 15-60,15-50,15-40,15-30,15-25 or 19-25 nucleotides in length, and the double stranded siRNA is about 15-60,15-50,15-40,15-30, 15-25 or 19-25 base pairs in length). siRNA duplexes may comprise 3′ overhangs of about 1 to about 4 nucleotides, alternatively about 2 to 3 nucleotides and 5′ phosphate termini. In some aspects, the siRNA lacks a terminal phosphate. In some aspects, one or both ends of siRNAs can include single-stranded 3′ overhangs that are two or three nucleotides in length, such as, for example, deoxythymidine (dTdT) or uracil (UU) that are not complementary to the target sequence. In some aspects, siRNA molecules can include nucleotide analogs (e.g., thiophosphate or G-clamp nucleotide analogs), alternative base linkages (e.g., phosphorothioate, phosphonoacetate, or thiophosphonoacetate) and other modifications useful for enhanced nuclease resistance, enhanced duplex stability, enhanced cellular uptake, or cell targeting.

In an aspect, the WETs of this disclosure are prepared into a composition or formulation for administration to a subject. The terms “composition” and “formulation” as used herein refer to their generally accepted meanings in the art. These terms generally refer to forms in which an agent is in a form suitable for administration (e.g., systemic or local administration) into a cell or to a subject, for example, a human, such as in a pharmaceutically acceptable carrier or diluent. Suitable forms, in part, depend upon the use or the route of entry, for example oral, transdermal, inhalation, or by injection. Such forms should not prevent the agent from reaching a target cell (i.e., a cell into which it is desirable to deliver the nucleic acid). For example, compositions injected into the blood stream should be soluble. Other factors for consideration in determining appropriate forms include considerations such as toxicity and forms that would prevent the agent from having its intended effect. Non-limiting examples of formulations and/or compositions for use with the materials of the instant present disclosure include: Lipid Nanoparticles (see for example Semple et al., 2010, Nat Biotechnol., February; 28(2): 172-6); P-glycoprotein inhibitors (such as Pluronic P85); biodegradable polymers, such as poly (DL-lactide-coglycolide) microspheres for sustained release delivery (Emerich, D F et al, 1990, Cell Transplant, 8, 47-58); and loaded nanoparticles, such as those made of polybutylcyanoacrylate. Other non-limiting examples of delivery strategies for the WETs of the present disclosure include those materials described in Boado et al., 1998, J. Pharm. Sci., 87,1308-1315; Tyler et al., 1999, FEBS Lett., 421,280-284; Partridge et al., 1995, PNAS USA., 92,5592-5596; Boado, 1995, Adv. Drug Delivery Rev., 15,73-107; Aldrian-Herrada et al., 1998, Nucleic Acids Res., 26,4910-4916; and Tyler et al., 1999, PNAS USA., 96, 7053-7058. A “pharmaceutically acceptable composition” or “pharmaceutically acceptable formulation” can refer to a form that allows for the effective distribution of the WET of the instant disclosure to the physical location most-suitable for their desired activity.

In an aspect, the composition and/or formulation may contain additional ingredients. As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials.

Administration of one or more WETs in accordance with the present disclosure may be continuous or intermittent, depending, for example, upon the recipient's physiological condition. whether the purpose of the administration is therapeutic or prophylactic, and other factors known to skilled practitioners. The administration of one or more WETs may be essentially continuous over a preselected period of time or may be in a series of spaced doses. Both local and systemic administration is contemplated. Conventional methods, known to those of ordinary skill in the art of medicine, can be used to administer of one or more WETs to a mammalian subject. The of one or more WETs can be administered via oral, subcutaneous, intravenous, intrapulmonary, transmucosal, intraperitoneal, intrauterine, sublingual, intrathecal, or intramuscular routes.

The amount of antibody useful in the disclosed methods in the pharmaceutical composition of the present disclosure will depend upon the nature and severity of the condition being treated, and on the nature of prior treatments that the patient has undergone. Ultimately, the attending physician will decide the amount of protein of the present disclosure with which to treat each individual patient. It is to be expected that the dosage will vary according to the age, weight and response of the individual patient. Initially, the attending physician will administer low doses of antibodies of the present methods and observe the patient's response. Larger doses of antibodies of the present disclosure may be administered until the optimal therapeutic effect is obtained for the patient, and at that point the dosage is not increased further. It is contemplated that the various pharmaceutical compositions used to practice the methods herein should contain about 0.01 μg to about 100 mg (alternatively about 0.1 μg to about 10 mg, alternatively about 0.1 μg to about 1 mg) of antibody of the present disclosure per kg body weight. When administered, the therapeutic composition for use in this disclosure is, of course, in a pyrogen-free, physiologically acceptable form. Therapeutically useful agents that may also optionally be included in the WETs of the preset disclosure as described above, may alternatively or additionally, be administered simultaneously or sequentially with the WETs in the methods of the disclosure. The exact formulation, route of administration and dosage can be chosen by the individual physician in view of the patient's condition. See, e.g., Fingl et al., THE PHARMACOLOGICAL BASIS OF THERAPEUTICS (latest edition). Dosage amount and interval may be adjusted individually to provide plasma levels of the active moiety sufficient to maintain the desired therapeutic effects, or minimal effective concentration (MEC), The MEC will vary for each compound but can be estimated from in vitro data; for example, the concentration necessary to achieve 50-90% inhibition of biologic activity using the assays described herein. The antibody provided herein can be administered alone or in combination with other therapeutic modalities.

The following items are exemplary, but not limiting, of the instantly inventive compositions, methods, and uses disclosed herein.
1. A composition comprising two materials selected from a material group, the material group consisting of (a) a neuronal nitric oxide synthase silencing nucleotide; (b) a neuronal nitric oxide synthase binding antibody; (c) an endothelial nitric oxide synthase silencing nucleotide; (d) an endothelial nitric oxide synthase binding antibody; (e) an inducible nitric oxide synthase silencing nucleotide; (f) an inducible nitric oxide synthase binding antibody; (g) a calcitonin gene-related peptide silencing nucleotide; (h) a calcitonin gene-related peptide binding antibody; (i) a disintegrin and metalloprotease peptide silencing nucleotide; (j) a disintegrin and metalloprotease binding antibody; (k) a bradykinin peptide silencing nucleotide; (1) a bradykinin binding antibody; (m) a substance P peptide silencing nucleotide; (n) a substance P binding antibody; (o) a histidine decarboxylase silencing nucleotide; (p) a histidine decarboxylase binding antibody; (q) an antihistamine; (r) a bradykinin antagonist; (s) a cyclooxygenase silencing nucleotide; (t) a cyclooxygenase binding antibody; (u) a small molecule prostacyclin inhibitor; (v) a prostacyclin synthase silencing nucleotide; and (w) a prostacyclin synthase binding antibody.
2. The composition of item 1, wherein the composition comprises three materials selected from the material group.
3. The composition of item 1, wherein the composition comprises four materials selected from the material group.
4. The composition of item 1, wherein the composition comprises five materials selected from the material group.
5. The composition of item 1, wherein the composition comprises six materials selected from the material group.
6. The composition of item 1, wherein the composition comprises seven materials selected from the material group.
7. The composition of item 1, wherein the composition comprises eight materials selected from the material group.
9. The composition of item 1, wherein the composition comprises ten materials selected from the material group.
10. The composition of item 1, wherein the composition comprises eleven materials selected from the material group.
11. The composition of item 1, wherein the composition comprises twelve materials selected from the material group.
12. The composition of item 1, wherein the composition comprises thirteen materials selected from the material group.
13. The composition of item 1, wherein the composition comprises fourteen materials selected from the material group.
14. The composition of item 1, wherein the composition comprises fifteen materials selected from the material group.
15. The composition of item 1, wherein the composition comprises sixteen materials selected from the material group.
16. The composition of item 1, wherein the composition comprises seventeen materials selected from the material group.
17. The composition of item 1, wherein the composition comprises eighteen materials selected from the material group.
18. The composition of item 1, wherein the composition comprises nineteen materials selected from the material group.
19. The composition of item 1, wherein the composition comprises twenty materials selected from the material group.
20. The composition of item 1, wherein the composition comprises twenty-one materials selected from the material group.
21. The composition of item 1, wherein the composition comprises twenty-two materials selected from the material group.
22. The composition of item 1, wherein the composition comprises all the materials selected from the material group.
23. The composition of item 1, wherein at least one of the (b), (d), (f), (h), (l), (n), (p), (t) or (w) materials is from the IgG family of immunoglobulins.
24. The composition of item 2, wherein at least one of the (b), (d), (f), (h), (l), (n), (p), (t) or (w) materials is from the IgG family of immunoglobulins.
25. The composition of item 3, wherein at least one of the (b), (d), (f), (h), (l), (n), (p), (t) or (w) materials is from the IgG family of immunoglobulins.
26. The composition of item 4, wherein at least one of the (b), (d), (f), (h), (l), (n), (p), (t) or (w) materials is from the IgG family of immunoglobulins.
27. The composition of item 5, wherein at least one of the (b), (d), (f), (h), (l), (n), (p), (t) or (w) materials is from the IgG family of immunoglobulins.
28. The composition of item 6, wherein at least one of the (b), (d), (f), (h), (l), (n), (p), (t) or (w) materials is from the IgG family of immunoglobulins.
29. The composition of item 7, wherein at least one of the (b), (d), (f), (h), (l), (n), (p), (t) or (w) materials is from the IgG family of immunoglobulins.
30. The composition of item 8, wherein at least one of the (b), (d), (f), (h), (l), (n), (p), (t) or (w) materials is from the IgG family of immunoglobulins.
31. The composition of item 9, wherein at least one of the (b), (d), (f), (h), (l), (n), (p), (t) or (w) materials is from the IgG family of immunoglobulins.
32. The composition of item 10, wherein at least one of the (b), (d), (f), (h), (l), (n), (p), (t) or (w) materials is from the IgG family of immunoglobulins.
33. The composition of item 11, wherein at least one of the (b), (d), (f), (h), (l), (n), (p), (t) or (w) materials is from the IgG family of immunoglobulins.
34. The composition of item 12, wherein at least one of the (b), (d), (f), (h), (l), (n), (p), (t) or (w) materials is from the IgG family of immunoglobulins.
35. The composition of item 13, wherein at least one of the (b), (d), (f), (h), (l), (n), (p), (t) or (w) materials is from the IgG family of immunoglobulins.
36. The composition of item 14, wherein at least one of the (b), (d), (f), (h), (l), (n), (p), (t) or (w) materials is from the IgG family of immunoglobulins.
37. The composition of item 15, wherein at least one of the (b), (d), (f), (h), (l), (n), (p), (t) or (w) materials is from the IgG family of immunoglobulins.
38. The composition of item 16, wherein at least one of the (b), (d), (f), (h), (l), (n), (p), (t) or (w) materials is from the IgG family of immunoglobulins.
39. The composition of item 17, wherein at least one of the (b), (d), (f), (h), (l), (n), (p), (t) or (w) materials is from the IgG family of immunoglobulins.
40. The composition of item 18, wherein at least one of the (b), (d), (f), (h), (l), (n), (p), (t) or (w) materials is from the IgG family of immunoglobulins.
41. The composition of item 19, wherein at least one of the (b), (d), (f), (h), (l), (n), (p), (t) or (w) materials is from the IgG family of immunoglobulins.
42. The composition of item 20, wherein at least one of the (b), (d), (f), (h), (l), (n), (p), (t) or (w) materials is from the IgG family of immunoglobulins.
43. The composition of item 21, wherein at least one of the (b), (d), (f), (h), (l), (n), (p), (t) or (w) materials is from the IgG family of immunoglobulins.
44. The composition of item 22, wherein at least one of the (b), (d), (f), (h), (l), (n), (p), (t) or (w) materials is from the IgG family of immunoglobulins.
45. The composition of item 23 wherein at least one of the (b), (d), (f), (h), (l), (n), (p), (t) or (w) materials is a functional fragment.
46. The composition of item 24 wherein at least one of the (b), (d), (f), (h), (l), (n), (p), (t) or (w) materials is a functional fragment.
47. The composition of item 25 wherein at least one of the (b), (d), (f), (h), (l), (n), (p), (t) or (w) materials is a functional fragment.
48. The composition of item 26 wherein at least one of the (b), (d), (f), (h), (l), (n), (p), (t) or (w) materials is a functional fragment.
49. The composition of item 27 wherein at least one of the (b), (d), (f), (h), (l), (n), (p), (t) or (w) materials is a functional fragment.
50. The composition of item 28 wherein at least one of the (b), (d), (f), (h), (l), (n), (p), (t) or (w) materials is a functional fragment.
51. The composition of item 29 wherein at least one of the (b), (d), (f), (h), (l), (n), (p), (t) or (w) materials is a functional fragment.
52. The composition of item 30 wherein at least one of the (b), (d), (f), (h), (l), (n), (p), (t) or (w) materials is a functional fragment.
53. The composition of item 31 wherein at least one of the (b), (d), (f), (h), (l), (n), (p), (t) or (w) materials is a functional fragment..
54. The composition of item 32 wherein at least one of the (b), (d), (f), (h), (l), (n), (p), (t) or (w) materials is a functional fragment.
55. The composition of item 33 wherein at least one of the (b), (d), (f), (h), (l), (n), (p), (t) or (w) materials is a functional fragment.
56. The composition of item 34 wherein at least one of the (b), (d), (f), (h), (l), (n), (p), (t) or (w) materials is a functional fragment..
57. The composition of item 35 wherein at least one of the (b), (d), (f), (h), (l), (n), (p), (t) or (w) materials is a functional fragment.
58. The composition of item 36 wherein at least one of the (b), (d), (f), (h), (l), (n), (p), (t) or (w) materials is a functional fragment.
59. The composition of item 37 wherein at least one of the (b), (d), (f), (h), (l), (n), (p), (t) or (w) materials is a functional fragment.
60. The composition of item 38 wherein at least one of the (b), (d), (f), (h), (l), (n), (p), (t) or (w) materials is a functional fragment.
61. The composition of item 39 wherein at least one of the (b), (d), (f), (h), (l), (n), (p), (t) or (w) materials is a functional fragment.
62. The composition of item 40 wherein at least one of the (b), (d), (f), (h), (l), (n), (p), (t) or (w) materials is a functional fragment.
63. The composition of item 41 wherein at least one of the (b), (d), (f), (h), (l), (n), (p), (t) or (w) materials is a functional fragment.
64. The composition of item 42 wherein at least one of the (b), (d), (f), (h), (l), (n), (p), (t) or (w) materials is a functional fragment.
65. The composition of item 43 wherein at least one of the (b), (d), (f), (h), (l), (n), (p), (t) or (w) materials is a functional fragment.
66. The composition of item 44 wherein at least one of the (b), (d), (f), (h), (l), (n), (p), (t) or (w) materials is a functional fragment.
67. A medicament comprising a composition, wherein the composition comprises two materials selected from a material group, the material group consisting of (a) a neuronal nitric oxide synthase silencing nucleotide; (b) a neuronal nitric oxide synthase binding antibody; (c) an endothelial nitric oxide synthase silencing nucleotide; (d) an endothelial nitric oxide synthase binding antibody; (e) an inducible nitric oxide synthase silencing nucleotide; (f) an inducible nitric oxide synthase binding antibody; (g) a calcitonin gene-related peptide silencing nucleotide; (h) a calcitonin gene-related peptide binding antibody; (i) a disintegrin and metalloprotease peptide silencing nucleotide; (j) a disintegrin and metalloprotease binding antibody; (k) a bradykinin peptide silencing nucleotide; (l) a bradykinin binding antibody; (m) a substance P peptide silencing nucleotide; (n) a substance P binding antibody; (o) a histidine decarboxylase silencing nucleotide; (p) a histidine decarboxylase binding antibody; (q) an antihistamine; (r) a bradykinin antagonist; (s) a cyclooxygenase silencing nucleotide; (t) a cyclooxygenase binding antibody; (u) a small molecule prostacyclin inhibitor; (v) a prostacyclin synthase silencing nucleotide; and (w) a prostacyclin synthase binding antibody.
68. The medicament of item 67, wherein the composition comprises three materials selected from the material group.
69. The medicament of item 67, wherein the composition comprises four materials selected from the material group.
70. The medicament of item 67, wherein the composition comprises five materials selected from the material group.
71. The medicament of item 67, wherein the composition comprises six materials selected from the material group.
72. The medicament of item 67, wherein the composition comprises seven materials selected from the material group.
73. The medicament of item 67, wherein the composition comprises eight materials selected from the material group.
74. The medicament of item 67, wherein the composition comprises nine materials selected from the material group.
75. The medicament of item 67, wherein the composition comprises ten materials selected from the material group.
76. The medicament of item 67, wherein the composition comprises eleven materials selected from the material group.
77. The medicament of item 67, wherein the composition comprises twelve materials selected from the material group.
78. The medicament of item 67, wherein the composition comprises thirteen materials selected from the material group.
79. The medicament of item 67, wherein the composition comprises fourteen materials selected from the material group.
80. The medicament of item 67, wherein the composition comprises fifteen materials selected from the material group.
81. The medicament of item 67, wherein the composition comprises sixteen materials selected from the material group.
82. The medicament of item 67, wherein the composition comprises seventeen materials selected from the material group.
83. The medicament of item 67, wherein the composition comprises eighteen materials selected from the material group.
84. The medicament of item 67, wherein the composition comprises nineteen materials selected from the material group.
85. The medicament of item 67, wherein the composition comprises twenty materials selected from the material group.
86. The medicament of item 67, wherein the composition comprises twenty-one materials selected from the material group.
87. The medicament of item 67, wherein the composition comprises twenty-two materials selected from the material group.
88. The medicament of item 67, wherein the composition comprises all of the materials from the material group.
89. A method comprising the administration of the medicament of item 67 to a subject having a clinical presentation of sepsis.
90. A method comprising the administration of the medicament of item 68 to a subject having a clinical presentation of sepsis.
91. A method comprising the administration of the medicament of item 69 to a subject having a clinical presentation of sepsis.
92. A method comprising the administration of the medicament of item 70 to a subject having a clinical presentation of sepsis.
93. A method comprising the administration of the medicament of item 71 to a subject having a clinical presentation of sepsis.
94. A method comprising the administration of the medicament of item 72 to a subject having a clinical presentation of sepsis.
95. A method comprising the administration of the medicament of item 73 a subject having a clinical presentation of sepsis.
96. A method comprising the administration of the medicament of item 74 to a subject having a clinical presentation of sepsis.
97. A method comprising the administration of the medicament of item 75 to a subject having a clinical presentation of sepsis.
98. A method comprising the administration of the medicament of item 76 to a subject having a clinical presentation of sepsis.
99. A method comprising the administration of the medicament of item 77 to a subject having a clinical presentation of sepsis.
100. A method comprising the administration of the medicament of item 78 to a subject having a clinical presentation of sepsis.
101. A method comprising the administration of the medicament of item 79 to a subject having a clinical presentation of sepsis.
102. A method comprising the administration of the medicament of item 80 to a subject having a clinical presentation of sepsis.
103. A method comprising the administration of the medicament of item 81 to a subject having a clinical presentation of sepsis.
104. A method comprising the administration of the medicament of item 82 to a subject having a clinical presentation of sepsis.
105. A method comprising the administration of the medicament of item 83 to a subject having a clinical presentation of sepsis.
106. A method comprising the administration of the medicament of item 84 to a subject having a clinical presentation of sepsis.
107. A method comprising the administration of the medicament of item 85 to a subject having a clinical presentation of sepsis.
108. A method comprising the administration of the medicament of item 86 to a subject having a clinical presentation of sepsis.
109. A method comprising the administration of the medicament of item 87 to a subject having a clinical presentation of sepsis.
110. A method comprising the administration of the medicament of item 88 to a subject having a clinical presentation of sepsis.
111. Use of the medicament of item 67 to reduce expression of sepsis-related pathogenic compounds.
112. Use of the medicament of item 68 to reduce expression of sepsis-related pathogenic compounds.
113. Use of the medicament of item 69 to reduce expression of sepsis-related pathogenic compounds.
114. Use of the medicament of item 70 to reduce expression of sepsis-related pathogenic compounds.
115. Use of the medicament of item 71 to reduce expression of sepsis-related pathogenic compounds.
116. Use of the medicament of item 72 to reduce expression of sepsis-related pathogenic compounds.
117. Use of the medicament of item 73 to reduce expression of sepsis-related pathogenic compounds.
118. Use of the medicament of item 74 to reduce expression of sepsis-related pathogenic compounds.
119. Use of the medicament of item 75 to reduce expression of sepsis-related pathogenic compounds.
120. Use of the medicament of item 76 to reduce expression of sepsis-related pathogenic compounds.
121. Use of the medicament of item 77 to reduce expression of sepsis-related pathogenic compounds.
122. Use of the medicament of item 78 to reduce expression of sepsis-related pathogenic compounds.
123. Use of the medicament of item 79 to reduce expression of sepsis-related pathogenic compounds.
124. Use of the medicament of item 80 to reduce expression of sepsis-related pathogenic compounds.
125. Use of the medicament of item 81 to reduce expression of sepsis-related pathogenic compounds.
126. Use of the medicament of item 82 to reduce expression of sepsis-related pathogenic compounds.
127. Use of the medicament of item 83 to reduce expression of sepsis-related pathogenic compounds.
128. Use of the medicament of item 84 to reduce expression of sepsis-related pathogenic compounds..
129. Use of the medicament of item 85 to reduce expression of sepsis-related pathogenic compounds.
130. Use of the medicament of item 86 to reduce expression of sepsis-related pathogenic compounds.
131. Use of the medicament of item 87 to reduce expression of sepsis-related pathogenic compounds.
132. Use of the medicament of item 88 to reduce expression of sepsis-related pathogenic compounds.

In some embodiments, use of the compositions and medicaments disclosed herein occur in vitro, In some embodiments, use of the compositions and medicaments disclosed herein occur in vivo.

It will be apparent to those skilled in the art that various modifications and variations can be made in the present disclosure without departing from the scope or spirit of the disclosure, Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1-47. (canceled)

48. A composition comprising two materials selected from a material group, the material group consisting of (a) a neuronal nitric oxide synthase silencing nucleotide; (b) a neuronal nitric oxide synthase binding antibody; (c) an endothelial nitric oxide synthase silencing nucleotide; (d) an endothelial nitric oxide synthase binding antibody; (e) an inducible nitric oxide synthase silencing nucleotide; (f) an inducible nitric oxide synthase binding antibody; (g) a calcitonin gene-related peptide silencing nucleotide; (h) a calcitonin gene-related peptide binding antibody; (i) a disintegrin and metalloprotease peptide silencing nucleotide; (j) a disintegrin and metalloprotease binding antibody; (k) a bradykinin peptide silencing nucleotide; (l) a bradykinin binding antibody; (m) a substance P peptide silencing nucleotide; (n) a substance P binding antibody; (o) a histidine decarboxylase silencing nucleotide; (p) a histidine decarboxylase binding antibody; (q) an antihistamine; (r) a bradykinin antagonist; (s) a cyclooxygenase silencing nucleotide; (t) a cyclooxygenase binding antibody; (u) a small molecule prostacyclin inhibitor; (v) a prostacyclin synthase silencing nucleotide; and (w) a prostacyclin synthase binding antibody.

49. The composition of claim 48, wherein the composition comprises three materials selected from the material group.

50. The composition of claim 48, wherein the composition comprises four materials selected from the material group.

51. The composition of claim 48, wherein the composition comprises five materials selected from the material group.

52. The composition of claim 48, wherein the composition comprises six materials selected from the material group.

53. The composition of claim 48, wherein the composition comprises seven materials selected from the material group.

54. The composition of claim 48, wherein the composition comprises eight materials selected from the material group.

55. The composition of claim 48, wherein the composition comprises nine materials selected from the material group.

56. The composition of claim 48, wherein the composition comprises ten materials selected from the material group.

57. The composition of claim 48, wherein the composition comprises eleven materials selected from the material group.

58. The composition of claim 48, wherein the composition comprises twelve materials selected from the material group.

59. The composition of claim 48, wherein the composition comprises thirteen materials selected from the material group.

60. The composition of claim 48, wherein the composition comprises fourteen materials selected from the material group.

61. The composition of claim 48, wherein the composition comprises fifteen materials selected from the material group.

62. The composition of claim 48, wherein the composition comprises sixteen materials selected from the material group.

63. The composition of claim 48, wherein the composition comprises seventeen materials selected from the material group.

64. The composition of claim 48, wherein the composition comprises twenty-three materials selected from the material group.

65. The composition of claim 48, wherein at least one of the (b), (d), (f), (h), (1), (n), (p), (t) or (w) materials is from the IgG family of immunoglobulins.

66. A medicament comprising a composition, wherein the composition comprises two materials selected from a material group, the material group consisting of (a) a neuronal nitric oxide synthase silencing nucleotide; (b) a neuronal nitric oxide synthase binding antibody; (c) an endothelial nitric oxide synthase silencing nucleotide; (d) an endothelial nitric oxide synthase binding antibody; (e) an inducible nitric oxide synthase silencing nucleotide; (f) an inducible nitric oxide synthase binding antibody; (g) a calcitonin gene-related peptide silencing nucleotide; (h) a calcitonin gene-related peptide binding antibody; (i) a disintegrin and metalloprotease peptide silencing nucleotide; (j) a disintegrin and metalloprotease binding antibody; (k) a bradykinin peptide silencing nucleotide; (l) a bradykinin binding antibody; (m) a substance P peptide silencing nucleotide; (n) a substance P binding antibody; (o) a histidine decarboxylase silencing nucleotide; (p) a histidine decarboxylase binding antibody; (q) an antihistamine; (r) a bradykinin antagonist; (s) a cyclooxygenase silencing nucleotide; (t) a cyclooxygenase binding antibody; (u) a small molecule prostacyclin inhibitor; (v) a prostacyclin synthase silencing nucleotide; and (w) a prostacyclin synthase binding antibody.

67. A method comprising the administration of a medicament to a subject having a clinical presentation of sepsis, wherein the medicament comprises a composition, wherein the composition comprises two materials selected from a material group, the material group consisting of (a) a neuronal nitric oxide synthase silencing nucleotide; (b) a neuronal nitric oxide synthase binding antibody; (c) an endothelial nitric oxide synthase silencing nucleotide; (d) an endothelial nitric oxide synthase binding antibody; (e) an inducible nitric oxide synthase silencing nucleotide; (f) an inducible nitric oxide synthase binding antibody; (g) a calcitonin gene-related peptide silencing nucleotide; (h) a calcitonin gene-related peptide binding antibody; (i) a disintegrin and metalloprotease peptide silencing nucleotide; (j) a disintegrin and metalloprotease binding antibody; (k) a bradykinin peptide silencing nucleotide; (l) a bradykinin binding antibody; (m) a substance P peptide silencing nucleotide; (n) a substance P binding antibody; (o) a histidine decarboxylase silencing nucleotide; (p) a histidine decarboxylase binding antibody; (q) an antihistamine; (r) a bradykinin antagonist; (s) a cyclooxygenase silencing nucleotide; (t) a cyclooxygenase binding antibody; (u) a small molecule prostacyclin inhibitor; (v) a prostacyclin synthase silencing nucleotide; and (w) a prostacyclin synthase binding antibody.
